(19) United States
(12) Patent Application Publication
(10) Pub. No.: US 2010/0168770 A1
(43) Pub. Date: Jul. 1, 2010

(54) LAPAROSCOPIC METHOD FOR SUTURING IN A BODY CAVITY

(75) Inventors: Richard S. Wilkenfeld, Montgomery, TX (US); Rhoda Wilkenfeld, Boca Raton, FL (US)

Correspondence Address: BUSKOP LAW GROUP, P.C., 4511 Dacoma Street, HOUSTON, TX, 77092, US

(73) Assignee: Richard S. Wilkenfeld, Conroe, TX (US)

(21) Appl. No.: 12/400,413

(22) Filed: Mar. 9, 2009

Related U.S. Application Data
(63) Continuation-in-part of application No. 12/346,046, filed on Dec. 30, 2008.

(51) Int. Cl. A61B 17/062

(52) U.S. Cl. 606/148

(57) ABSTRACT

The present embodiments relate to a laparoscopic method for suturing in a patient, namely by pushing at least one throw of a knot into a body cavity in a manner that is quick and easier on the patient, particularly lap band patients.

### CROSS REFERENCE TO RELATED APPLICATIONS

The present application is a Continuation in Part of co-pending U.S. patent application Ser. No. 12/346,046 filed on Dec. 30, 2008, entitled “Laparoscopic Knot Pushing Device” and is incorporated herein in its entirety.

### FIELD

The present embodiments generally relate to a laparoscopic method for suturing in a patient, namely by pushing at least one throw of a knot into a body cavity in a manner that is quick and easier on the patient, particularly lap band patients.

### BACKGROUND

A need exists for a method for suturing a patient through a narrow catheter or trocar that is quick.

A need exists for a method for easily making and pushing throws for forming knots to proximate internal tissues in a body cavity.

A need exists for a method to push throws of a knot down into a patient's body cavity for a surgeon to quickly tie off a suture stitch.

A further need exists for a method that makes it easy for surgeon or less skilled health professional to manipulate, suture threads and slide knots that are not slip knots into a patient's body cavity with a minimum diameter trocar, invoking less stress on the patient as compared with larger diameter devices, in a laparoscopic manner, without the fingers of the heath professional entering the body cavity.

The present embodiments meet these needs.

### BRIEF DESCRIPTION OF THE DRAWINGS

The detailed description will be better understood in conjunction with the accompanying drawings as follows:

FIG. 1 is a top view of an embodiment of a laparoscopic knot pushing device usable in the present method.

FIG. 2 is a detail of the planar face and hole of the laparoscopic knot tying device of FIG. 1

FIG. 3 shows a detail of a hole in the planar face of FIG. 2.

FIG. 4 depicts a camera and targeting light mounted to the elongated body of the knot pushing device usable in the method of the invention.

FIGS. 5A-5C shows a diagram depicting the steps of an embodiment of the present method.

The present embodiments are detailed below with reference to the listed Figures.

### DETAILED DESCRIPTION OF THE EMBODIMENTS

Before explaining the present method in detail, it is to be understood that the method is not limited to the particular embodiments and that it can be practiced or carried out in various ways.

The present embodiments relate to a method of tying off sutures for internal body tissues in a body cavity.

The method can involve inserting a trocar, which can have an annulus, through a body wall into a body cavity. For example, a 5 millimeter diameter trocar can be inserted through an abdomen wall to reach stomach tissues.

The suture can then be passed down the annulus of the trocar while the trocar is in the body cavity to a first piece and a second piece of internal body tissue in the body cavity. For example, the suture can be connected to a needle that can be slide down the annulus to the stomach tissues. The needle can penetrate the first internal body tissue, such as a first portion of the outer wall of the stomach, and then the needle can penetrate the second internal body tissue, such as a second portion. The first portion of internal body tissue can between about 1 millimeter of the second portion of internal body tissue as in the case of a opening in an organ or the first portion of internal body tissue can be about 3 centimeters to about 4 centimeters from the second portion of internal body tissue in the case of a lap band placement.

The needle, which can be a standard surgical needle, with the suture attached can be pulled back through the same trocar through which it was inserted and while pulling the needle through the trocar, the internal body tissue can be proximated together using gentle traction on both ends of the suture.

The needle can then be removed from the trocar. Then, the needle can be removed from the suture leaving a first end of suture and a second end of suture extending from the trocar opposite the body cavity. The needle can further be cut with scissors from the suture.

The method can continue by passing the first end of suture through a hole in a laparoscopic knot pushing device.

Next, at least one throw for forming a knot can be created around the second end of suture between a second end of the laparoscopic knot pushing device and the trocar.

Then, simultaneously while holding both the first end and the second end of the suture together, pushing the at least one throw down through the annulus of the trocar to the first piece and second piece of internal body tissue using the laparoscopic knot pushing device until the at least one throw is adjacent at least one of the internal body tissues further proximating together the first piece of internal body tissue with the second piece of internal body tissue in the body cavity using the suture.

The method can be used for suturing inside a body cavity, suturing organs or internal tissue, such as cartilage that is not easily accessible without making a big hole in a patient, or cutting the patient.

As part of this method, the laparoscopic device with suture passing through the hole can be pulled back through the annulus of the trocar while maintaining the trocar through the body wall of the internal body cavity. Typically, trocars stay in the patient during suturing until all steps of the suturing process are complete.

Next, an additional throw of the knot can be formed between the second end of the laparoscopic knot pushing device and the trocar, and simultaneously while holding both ends of the suture together, the second throw can then be pushed down through the trocar to the first piece and second piece of internal body tissue using the laparoscopic knot pushing device until the second throw is on top of the first throw.

This overlap of the first throw on the second throw can be critical for forming a secure knot. Adjacent throws do not work as well as the overlapping throws. Sometimes, up to about 4 throws can be used by a surgeon.

The sequence of steps can be repeated, as additional throws are needed with the making of the additional throw, pushing of the additional throw down the trocar, and withdrawing the laparoscopic knot pushing device until a surgeon determines the knot is secure.

The method can contemplate that the suture is a stitch. The suture can be a biodegradable filament that can be monofilament or braided multi-filament, such as PDS™ filament.

In another embodiment of the method, the suture can be made from a permanent multifilament or a permanent monofilament. Permanent monofilament can be Prolene™. Permanent multifilament can be silk, Ethibond™, Ethalon™, another polypropylene multifilament, another polyethylene multifilament, or combinations thereof. Other permanent multifilaments can be used, and combinations of these permanent multifilaments and permanent monofilaments can be used.

Some patients can require multiple sutures, which can involve about 48 inch original lengths of a suture, leaving in the patient, about 1 inch of suture material or less.

The method can be used when the two pieces of internal body tissue are adjacent each other, such as on either side of a cut, such as in replacement organ surgery, or can be proximate to each other, like parts of a stomach organ as in lap band surgery.

The method can contemplate that surgery using overlapping internal body tissue can be very effective.

An example of the overlapping of tissues can occur when a first piece of internal body tissue is a first outside portion of a stomach, and the second piece of internal body tissue is a second outside portion of a stomach, and the first and second outside portions of stomach are overlaid on top of each other, enabling the overlapped internal body tissue to support a device in position.

The device to be held in position can be a Lap band™, feeding tubes, catheters, or similar internal body cavity device.

The method can also contemplate that a first piece of internal body tissue can be a first outside portion of a stomach, and the second piece of internal body tissue can be a second outside portion of a stomach, and the first and second outside portions of stomach can be overlaid on top of each other, enabling the overlapped internal body tissue to provide reinforcement of the internal body tissues. So two different uses of the overlapping of the tissue structure can be contemplated by this method.

The method can be contemplated for use with internal body tissue such as organ internal body tissue. The internal body tissue can be hollow body internal body tissue or solid body internal body tissue. The tissue can be from the esophagus, stomach, small and large intestines, bladder, lungs, tendons, cartilage, liver, pancreatic, kidney, spleen, arteries, veins, or combinations thereof.

The method can be particularly usable when surgery requires that the internal body cavity has been inflated or expanded such as with carbon dioxide.

The method can contemplate that the trocar can be between about 2 inches to about 10 inches in length, and have an inner diameter forming the annulus between about 2 millimeters to about 15 millimeters.

The method can also contemplate the additional step of connecting a targeting light to the laparoscopic knot pushing device for illuminating a targeted area of the first piece and second piece of internal body tissue during the pushing of the throws of the knot. For example, a fiber optic cable can be used to provide the light of this device in this method.

The method can also contemplate the step of connecting a camera with the laparoscopic knot pushing device for close inspection and monitoring of the knot and the first piece and second pieces of internal body tissue.

The term “the internal body cavity” can refer to a joint cavity, an abdominal cavity, a bladder cavity, an oral cavity, or a thoracic cavity.

The at least one throw of the method can be formed from the suture and wherein the suture can have a first piece of suture that passes through the internal body tissue, a looped piece of suture that loops around the first piece of suture, and a second piece of suture that can be passed through the looped piece of suture, and wherein the looped piece of suture surrounds the first piece of suture, and wherein the first piece of suture, the looped piece of suture and the second piece of suture are integrally connected and contiguous with each other.

The method can further use a laparoscopic knot pushing device, which can have an elongated body having an elongated body axis, a first end and a second end. The first end can be adapted for gripping by a user. The device can further have a planar face formed in the second end and the planar face can be opposite the elongated body. The planar face can have a first side with a first beveled edge and a second beveled edge. The planar face can be positioned about 5 degrees to about 20 degrees from the axis of the elongated body. A hole can be in the planar face. The hole can have a top beveled edge and a bottom beveled edge, which can be disposed in the planar face and can be perpendicular to the elongated body axis.

The present embodiments can use a laparoscopic knot pushing device for lap band procedures or any other minimally invasive surgery procedures.

A benefit of this invention is that with the method, suturing becomes more secure than a prepared knot or a slip knot. This method can provide secure knots, known as surgical knots not slip knots. Surgical knots cab prevent tissue from becoming disengaged from the suture and each other. The use of surgical knots can enable faster healing by a patient because the tissue contact is maintained.

Another benefit of this invention is that the user can firmly work the method using the device preventing damage to adjacent tissue if the surgeon's hand should slip.

A benefit of the invention is that in the method use of a sterilizable device that is stiff, but slightly flexible can be used, allowing a secure knot to be inserted with a tight knot while simultaneously preventing the suture from breaking.

Still another benefit of the invention is that the method can be easier for surgeons to place and position knots that are secure knots than intracorporal knot tying. Sewing inside a body laparoscopically, with two needle holders is complex and difficult. This method allows interbody sewing with out of body knot tying. That is, the method allows a knot to be quickly and easily formed outside of the body and then the knot or portion of the knot (the “throw”) can be slid into the body, with a knot pushing device, making less complicated sewing than trying to tie a knot in the body itself.

The method can allow a surgeon to make knots in a larger volume of space outside of the body. The method can further allow the surgeons working in very small spaces to make secure surgical knots quickly, easily with less stress.

Another benefit of the invention is that the method takes less time to suture a patient than suturing without the knot tying device.

The method using the unique knot tying device can make surgery easier on a patient and enable the patient to spend less time under anesthesia. As an example, in a surgery using this method for knot tying, saves up to about 20 percent of the time for suturing by enabling the surgeon to tie knots outside of the body and then slip them into the body.

Another benefit of the invention is that the method allows a greater range of sutures to be used on a patient. This method can further allow increased versatility for a surgeon in the doctor's selection of sutures for a particular procedure. This method is particularly good for increasing versatility in the use of different types of sutures for laparoscopic surgeries. Now, sutures made of nylon, dissolving sutures, Dacron™ sutures, Neuralon™ sutures, Ethabond™ sutures and silk sutures can be easily used.

The method can enable a physician to perform surgeries on a patient faster than traditional surgeries, by at least 10 percent, which can reduce or eliminate diabetes in the patient.

The method can further enable a surgeon, to perform surgeries on a patient faster, than without the device, reducing the chance of tissues coming apart, where the surgeries are to reduce or eliminate high blood pressure in a patient undergoing lap band surgery.

The new method can enable a surgeon to repair hiatal hernias using native or synthetic tissues with less difficulty and greater security than non-suturing repairs such as tacking devices.

The method can enable a surgeon to perform surgeries on a patient to reduce or eliminate acid reflux very inexpensively, with less than about ½ the cost of performing the same surgery with a tacking device.

The method can be used on humans, horses, cows, or other mammals over about 15 pounds.

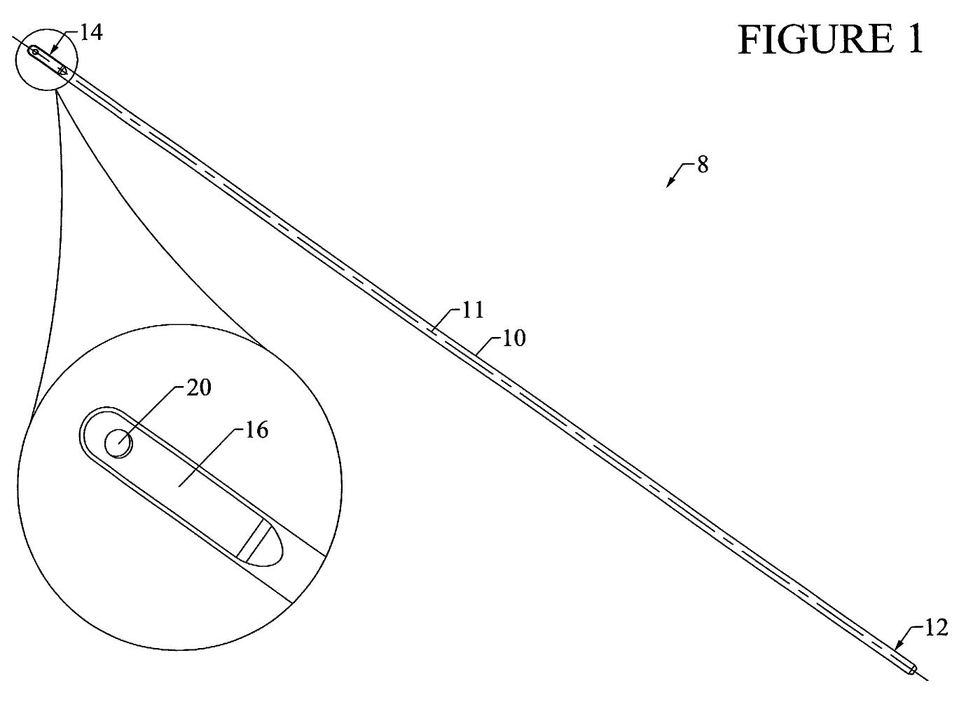

Turning now to FIG. 1, which depicts the laparoscopic knot pushing device (8) usable in the method. The device (8) has an elongated body (10) having an elongated body axis (11).

The elongated body can be between about 18 inches to about 24 inches long and have an overall diameter ranging from between about 3 millimeters to about 9.5 millimeters. The elongated body can have a planar face (16) with a hole (20). The elongated body is shown having a first end (12) for holding by a surgeon and a second end (14) with unique features.

In this embodiment, the elongated body can be made of stainless steel, or a sterilizable, non-deformable, impact resistant polymer, such as a polypropylene copolymer with polyethylene or a polypropylene with some polyvinyl chloride blended in. In another embodiment, the elongated body can be made of a stiff material having some flexibility such as a graphite composite or a polymer with between about 2 weight percent to about 20 weight percent of an elastomeric material blended in.

The elongated body can be sterilizable, such as with an autoclave or with ultraviolet light or sterilizable with chemicals and resist degradation during sterilization.

An embodiment of the device can be reused by a surgeon. A hollow embodiment can be reused as well as a solid version many times.

Another embodiment of the device can contemplate that a hollow, throw-away version of the device can be used, that is, a disposable device. A solid elongated body can be used in a disposable version as well. Disposable versions can have the advantage of being lightweight, easy to ship, and easy to store.

The elongated body can have an integral planar face or a removable planar face. The device can be an integral one-piece unit formed from a single piece of steel, such as surgical grade stainless steel.

The elongated body can have a diameter that fits within standard sized trocars.

If the device is a two-piece structure, the planar face can be threadably engaged with the elongated body, or it can be a forced fit into the body, such as a cavity formed in the end of the body.

The elongated body can also be made of polycarbonate or another autoclavable or sterilizable polymer that does not deform at high temperatures in an autoclave.

An embodiment can contemplate that the device can be about 100 percent metal, about 100 percent polymer or can be an autoclavable non-deformable polymer disposed over a metal cylindrical body.

An embodiment can contemplate that the elongated body can have a solid elongated body about 19 inches long, with body diameter of about 5 millimeters and a hole diameter of about 3 millimeters.

The shape of the elongated body can be rounded. The elongated body can be a rounded 8-sided structure, a rounded 4-sided structure, a rounded 6-sided structure, a cylinder structure or a conical structure.

The elongated body first end (12) can be adapted for gripping by a user, such as a lap band surgeon or another laparoscopic surgeon.

The device can be used for tying a knot after a suture is placed and pushing the knot down into a body through a cylindrical tube, such as a trocar. Trocars can vary in diameter from about 5 millimeters to about 15 millimeters and the device can have different diameters to fit within the different sized trocars. Sutures and the completed knot can then be used to connect patient tissue securely with this device.

FIG. 1 further shows planar face (16) can be formed in the second end (14). The planar face can be integral with the elongated body (10). The planar face (16) can have a hole (20) in line with the axis (11) of the elongated body, but can be formed perpendicular to the second end (14) axis of the body.

In an embodiment, the hole can be formed perpendicular to the axis of the elongated body or perpendicular to the second end (14).

The planar face can be tapered and can have a thickness ranging between about 2 millimeters to about 4 millimeters. However, if a larger diameter elongated body is used, the planar face can taper from about 2 millimeters to about 8 millimeters when the elongated body diameter is about 9 millimeters.

The width of the planar face can be up to about 9 millimeters. The hole in the planar face can have a diameter ranging from about 1.5 millimeters to about 7 millimeters. The elongated body can be tapered for a larger diameter at the first end to a smaller diameter by about 50 percent at the second end. The planar face can taper from the body that can be tapered to a diameter between about 20 percent to about 80 percent less than the diameter of the elongated body.

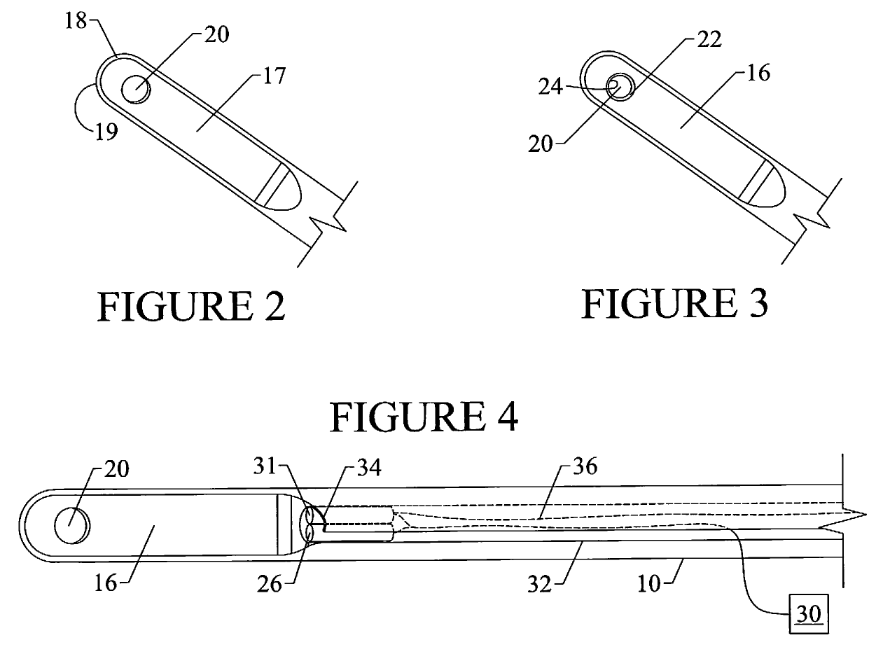

FIG. 2 shows a detail of the planar face having a first side (17) with a first rounded edge (18), a second rounded edge (19) opposite the first rounded edge, and the hole (20). Angled faces can cut the suture material, making it impossible to slide the newly tied knot into the body smoothly, so they are avoided with this device.

The planar face, in an embodiment can have the first and second rounded edges on at least a portion of the first side, that is the entire first side does not have to have rounded edges, just the distal tip of the device.

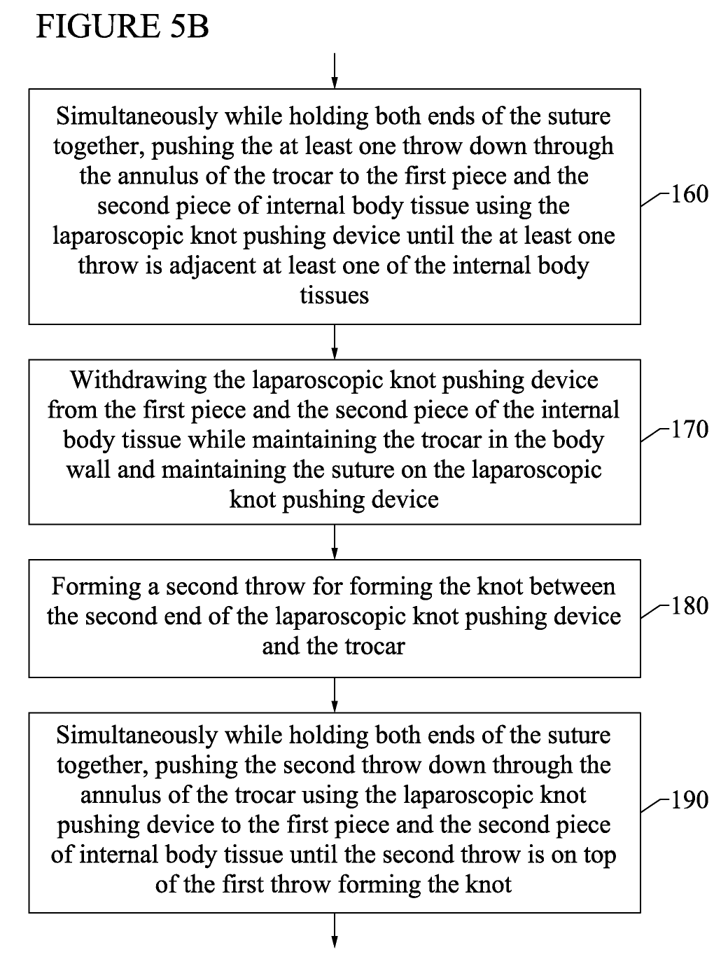

The planar face can be positioned from about 5 degrees to about 20 degrees from the axis of the elongated body (see FIG. 4). In an embodiment of the invention, the planar face can be between about 8 degrees to about 10 degrees from the axis of the elongated body.

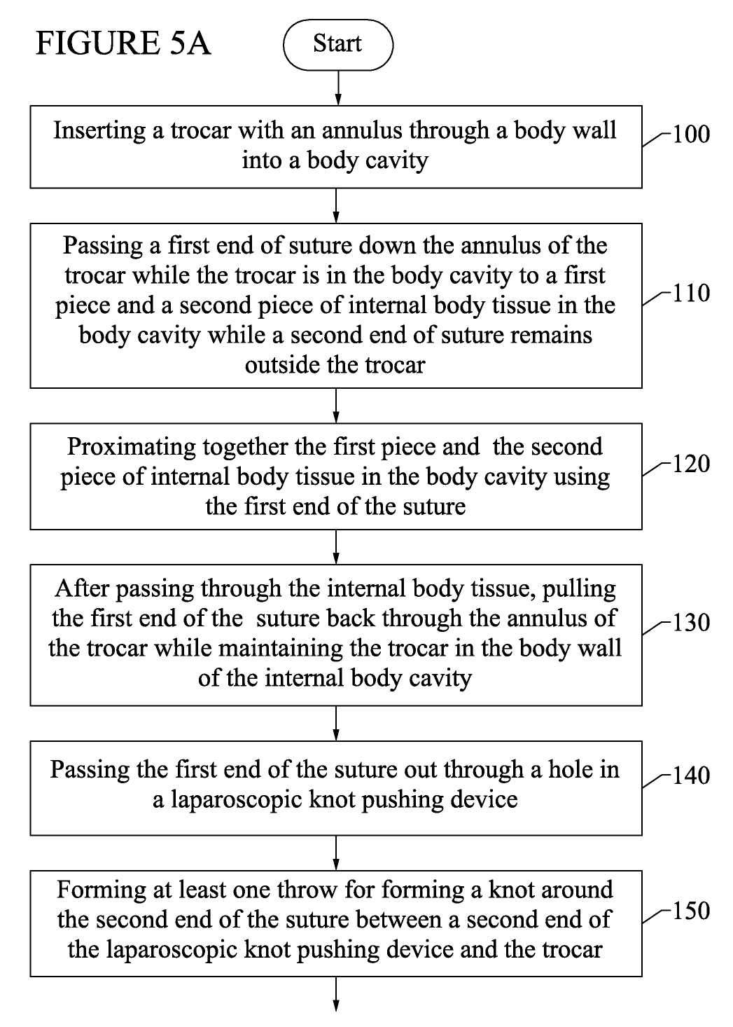

FIG. 3 shows a detail of the hole (20) with a top rounded edge (22) on the inside of the hole and a bottom rounded edge (24) on the inside of the hole disposed in the planar face (16). The hole can have a diameter from about 1.5 millimeters to about 7 millimeters.

The device can be machined from a surgical metal, such as stainless steel or another metal alloy that can sustain a corrosive environment.

The device can further be made from a molded material that withstands high temperatures without becoming brittle or cracking.

In an embodiment the device can include a non-slip coating disposed over the first end, such as a coating of sterilizable soft non-slip material, such as a synthetic rubber that can be between about 0.1 millimeters to about 0.5 millimeters in thickness.

In an operation on a patient, typically several trocars can be inserted into a patient through which suturing material can be placed to approximate tissues. Using the knot pushing device, a surgeon can take a suture and passes it down the trocar through the two tissues and brings it back out through the same trocar. The surgeon can then make a knot that can then be slid down the trocar that can secure the stitch thereby securely apposing the two tissues together.

Suture material from one end of the formed stitch can then be passed through the hole of the device. The surgeon can then form a throw of a knot between the second end of the device and the trocar. While holding both ends of the suture together, the surgeon can push the throw with the planar face of the device down to the tissues through the trocar while holding onto the first end of the device until the loop is secure. The surgeon can then withdraw the device without taking it off the end of the suture, and make another throw, again pushing the newly formed throw into the patient proximal to the initial throw, forming a knot. The process can then be repeated until the surgeon determines that the tissues are secure.

A benefit of the invention is that it is a “staple-free” method of connecting tissues using standard sutures that more surgeons are familiar with.

FIG. 4 shows an embodiment of the planar face (16), with hole (20), a targeting light (31), and a camera (26) secured to the elongated body (10). A power supply (30), which can be remote is shown in this Figure, connected to the camera (26) and targeting light (31). An embodiment contemplates that the elongated body (10) can be hollow and cables or wires for the camera and the targeting light can run down the center of the elongated body.

Although the camera and/or targeting light can be connected on the outside of the elongated body, FIG. 4 shows an embodiment using a channel (32), which can be disposed along the axis of the elongated body (10) for containing the targeting light (31), the camera (26), cable (36), or combinations thereof.

The channel can have rounded edges where the channel meets the outside of the elongated body. The channel can have a slight lip (34) or overhang over the channel formed as an integral part of the body to contain a cable (36) in the channel. The cable can be fiber optic cables.

The camera (26) and/or the targeting light (31) can be in the channel, and the cable (36) can be removably affixed to the camera, the targeting light, or combinations thereof.

An embodiment of the device can contemplate that the first end can be removably secured to the elongated body.

An embodiment of the device can further contemplate that the second end can be removably secured to the elongated body.

An embodiment of the invention can contemplate that the first end can have a reduced slip grip for a doctor, which can be formed by etching, scoring, cutting with a laser, by sputtering or in some other manner.

An embodiment can further contemplate that a non-stick coating, such as Teflon™ can be disposed over all or a portion of the second end of the device, to enhance smooth insertion of throws into the body.

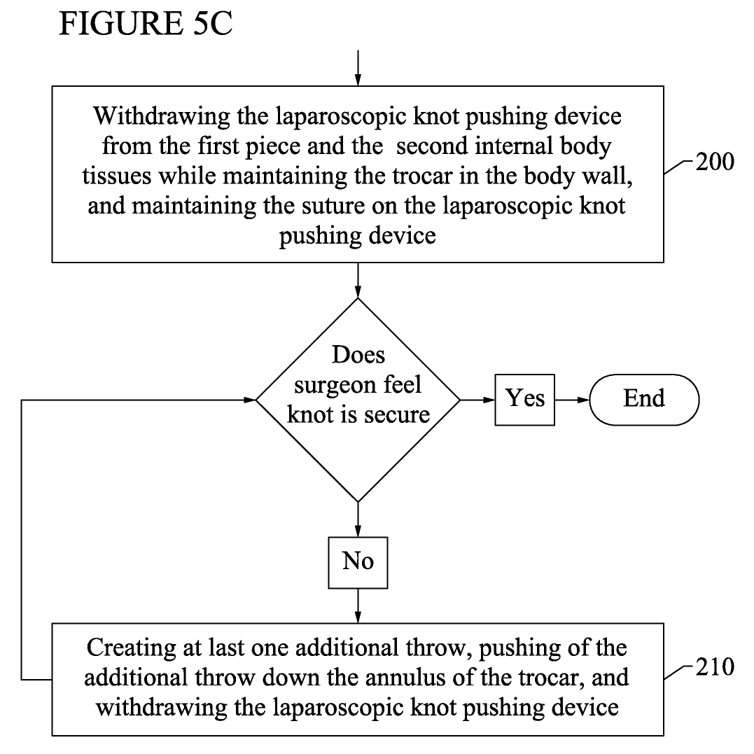

FIGS. 5A-5C shows a diagram depicting the steps of an embodiment of the present method.

A laparoscopic method of tying off sutures in a body cavity can comprise the following steps.

The first step of the method can be performed by, inserting a trocar with an annulus through a body wall into a body cavity (100).

The second step of the method can be performed by, passing a first end of suture down the annulus of the trocar while the trocar is in the body cavity to a first piece and a second piece of internal body tissue in the body cavity while a second end of suture remains outside the trocar (110).

The third step of the method can be performed by, proximating together the first piece and the second piece of internal body tissue in the body cavity using the first end of the suture (120).

The fourth step of the method can be performed by, after passing through the internal body tissue, pulling the first end of the suture back through the annulus of the trocar while maintaining the trocar in the body wall of the internal body cavity (130).

The fifth step of the method can be performed by, passing the first end of the suture out through a hole in a laparoscopic knot pushing device (140).

The sixth step of the method can be performed by, forming at least one throw for forming a knot around the second end of the suture between a second end of the laparoscopic knot pushing device and the trocar (150).

The seventh step of the method can be performed by, simultaneously while holding both ends of the suture together, pushing the at least one throw down through the annulus of the trocar to the first piece and the second piece of internal body tissue using the laparoscopic knot pushing device until the at least one throw is adjacent at least one of the internal body tissues (160).

The eighth step of the method can be performed by, withdrawing the laparoscopic knot pushing device from the first piece and the second piece of the internal body tissue while maintaining the trocar in the body wall and maintaining the suture on the laparoscopic knot pushing device (170).

The ninth step of the method can be performed by, forming a second throw for forming the knot between the second end of the laparoscopic knot pushing device and the trocar (180).

The tenth step of the method can be performed by, simultaneously while holding both ends of the suture together, pushing the second throw down through the annulus of the trocar using the laparoscopic knot pushing device to the first piece and the second piece of internal body tissue until the second throw is on top of the first throw forming the knot (190).

The eleventh step of the method can be performed by, withdrawing the laparoscopic knot pushing device from the first piece and the second internal body tissues while maintaining the trocar in the body wall, and maintaining the suture on the laparoscopic knot pushing device (200).

If the surgeon feels as though the knot is secure, then the method steps can be complete.

However, if the surgeon does not feel that the knot is secure, then the surgeon can repeat the optional twelfth step of, creating at last one additional throw, pushing of the additional throw down the annulus of the trocar, and withdrawing the laparoscopic knot pushing device (210), until the surgeon determines the knot is secure.

While these embodiments have been described with emphasis on the embodiments, it should be understood that within the scope of the appended claims, the embodiments might be practiced other than as specifically described herein.

What is claimed is:

1. A laparoscopic method of tying off sutures in a body cavity comprising the steps of:
a. inserting a trocar with an annulus through a body wall into a body cavity;
b. passing a first end of suture down the annulus of the trocar while the trocar is in the body cavity to a first piece and a second piece of internal body tissue in the body cavity while a second end of suture remains outside the trocar;
c. proximating together the first piece and the second piece of internal body tissue in the body cavity using the first end of the suture;
d. after passing through the internal body tissue, pulling the first end of the suture back through the annulus of the trocar while maintaining the trocar in the body wall of the internal body cavity;
e. passing the first end of the suture out through a hole in a laparoscopic knot pushing device;
f. forming at least one throw for forming a knot around the second end of the suture between a second end of the laparoscopic knot pushing device and the trocar;
g. simultaneously while holding both ends of the suture together, pushing the at least one throw down through the annulus of the trocar to the first piece and the second piece of internal body tissue using the laparoscopic knot pushing device until the at least one throw is adjacent at least one of the internal body tissues;
h. withdrawing the laparoscopic knot pushing device from the first piece and the second piece of the internal body tissue while maintaining the trocar in the body wall and maintaining the suture on the laparoscopic knot pushing device;
i. forming a second throw for forming the knot between the second end of the laparoscopic knot pushing device and the trocar;
j. simultaneously while holding both ends of the suture together, pushing the second throw down through the annulus of the trocar using the laparoscopic knot pushing device to the first piece and the second piece of internal body tissue until the second throw is on top of the first throw forming the knot; and
k. withdrawing the laparoscopic knot pushing device from the first piece and the second internal body tissues while maintaining the trocar in the body wall, and maintaining the suture on the laparoscopic knot pushing device; and
l. if needed, creating at last one additional throw, pushing of the additional throw down the annulus of the trocar, and withdrawing the laparoscopic knot pushing device until a surgeon determines the knot is secure.

2. The method of claim 1 wherein the suture is a stitch.

3. The method of claim 1, wherein the suture is biodegradable filament.

4. The method of claim 3, wherein the biodegradable filament is monofilament or braided multi-filament.

5. The method of claim 1, wherein the suture is permanent multifilament.

6. The method of claim 5, wherein the permanent multifilament comprises a member of the group consisting of: silk, ethibond™, ethalon™, prolene™, another polypropylene multifilament, another polyethylene multifilament or combinations thereof

7. The method of claim 1, wherein the two pieces of internal body tissue are adjacent each other.

8. The method of claim 1, wherein the two pieces of internal body tissue are overlapping each other.

9. The method of claim 8, wherein a first piece of internal body tissue is a first outside portion of a stomach, and the second piece of internal body tissue is a second outside portion of a stomach, and the first and second outside portions of stomach are overlaid on top of each other, enabling the overlapped internal body tissue to support a device in position.

10. The method of claim 8, wherein a first pieces of internal body tissue is a first outside portion of a stomach, and the second piece of internal body tissue is a second outside portion of a stomach, and the first and second outside portions of stomach are overlaid on top of each other, enabling the overlapped internal body tissue to provide reinforcement of the internal body tissues.

11. The method of claim 1, wherein the internal body tissue is organ internal body tissue,

12. The method of claim 11, wherein the internal body tissue is hollow body internal body tissue or solid body internal body tissue.

13. The method of claim 1, wherein the internal body cavity has been inflated or expanded.

14. The method of claim 13, wherein the internal body cavity is inflated with carbon dioxide.

15. The method of claim 1, wherein the trocar is between 2 inches to 10 inches in length, and has an inner diameter forming the annulus between 2 millimeters to 15 millimeters.

16. The method of claim 1, further comprising the step of connecting a targeting light to the laparoscopic knot pushing device for illuminating a targeted area of the first piece and the second piece of internal body tissue.

17. The method of claim 1, further comprising the step of connecting a camera with the laparoscopic knot pushing device for close inspection and monitoring of the knot and first and second pieces of internal body tissue.

18. The method of claim 1, wherein the internal body cavity comprises a joint cavity, a abdominal cavity, bladder cavity, oral cavity, or thoracic cavity.

19. The method of claim 1, wherein the at least one throw is formed from the suture and wherein the suture has a first piece of suture that passes through the internal body tissue, a looped piece of suture that loops around the first piece of suture, and a second piece of suture that is passed through the looped piece of suture, and wherein the looped piece of suture surrounds the first piece of suture, and wherein the first piece of suture, the looped piece of suture and the second piece of suture are integrally connected and contiguous with each other.

20. The method according to claim 1 wherein the laparoscopic knot pushing device comprises:
a. an elongated body having an elongated body axis and a first end and a second end, the first end adapted for gripping by a user;
b. a planar face formed in the second end wherein the planar face is opposite the elongated body, and wherein the planar face has a first side with a first beveled edge and a second beveled edge, and wherein the planar face is positioned 5 degrees to 20 degrees from the axis of the elongated body; and
c. a hole with a top beveled edge and a bottom beveled edge disposed in the planar face perpendicular to the elongated body axis.
